SEQUENCE LISTING (7 sequences)

SEQ ID NO: 1 - RNA, 22 nt, Homo sapiens
acucggcguggcgucggucgug

SEQ ID NO: 2 - DNA, 25 bp, Artificial Sequence
  Primer
ccagtccgccactgtactcacgacc

SEQ ID NO: 3 - DNA, 80 bp, Artificial Sequence
  Primer
agggttggaggttcatgtcaaggcccagtctcacgtattccactgaccaccagtcgcactgaggggactcggcgtggcgt

SEQ ID NO: 4 - DNA, 50 bp, Artificial Sequence
  Primer
tggaatacgtgagactgggcctttttttttagggttggaggttcatgtca

SEQ ID NO: 5 - DNA, 39 bp, Artificial Sequence
  Primer
ctgaccaccagtcgcactgagccagtccgccactgtact

SEQ ID NO: 6 - DNA, 11 bp, Artificial Sequence
  Primer
tggcgtcggtc

SEQ ID NO: 7 - DNA, 13 bp, Artificial Sequence
  Primer
cgtcggtcgtgag

(19) United States
(12) Patent Application Publication
(10) Pub. No.: US 2021/0189504 A1
(43) Pub. Date: Jun. 24, 2021

(54) METHOD AND KIT FOR MEASUREMENT OF RNA

(71) Applicant: KABUSHIKI KAISHA TOSHIBA, Tokyo (JP)

(72) Inventors: Koji HASHIMOTO, Atsugi (JP); Mika INADA, Tokyo (JP); Keiko ITO, Kawasaki (JP)

(73) Assignee: KABUSHIKI KAISHA TOSHIBA, Tokyo (JP)

(21) Appl. No.: 17/186,422

(22) Filed: Feb. 26, 2021

Related U.S. Application Data
(63) Continuation of application No. PCT/JP2020/008309, filed on Feb. 28, 2020.

(30) Foreign Application Priority Data
Jun. 14, 2019 (JP) 2019-111432

(51) Int. Cl. C12Q 1/6886

(52) U.S. Cl. CPC C12Q 1/6886

(57) ABSTRACT

According to the embodiments, miRNA having a nucleotide sequence represented by SEQ ID NO: 1 is used as a marker for cancer diagnosis. For example, a method for diagnosing cancer in a subject, comprising the step of: measuring the amount of RNA having the nucleotide sequence in a biological sample is disclosed.

### CROSS-REFERENCE TO RELATED APPLICATION(S)

This application is continuation application of International Application No. PCT/JP2020/008309, filed on Feb. 28, 2020, which claims priority to Japanese Patent Application No. 2019-111432 filed on Jun. 14, 2019, the entire contents of which are incorporated herein by reference.

### FIELD

Embodiments described herein relate generally to a method and kit for measurement of microRNA.

### BACKGROUND

Various techniques are used for cancer tests. Specific procedures include a method for capturing internal cancer from outside of the body as an image such as an X-ray test, ultrasonography, and the like; a method for observing the presence of cancerous cells in obtained specimens under a microscope (cytodiagnosis); and a method for measuring the amount of tumor markers in the blood; and the like. There may be difficulties in the diagnostic imaging modalities in the early detection of cancer because cancer cannot be detected until it has grown to a specific volume; it is hidden by other organs and cannot be found; cancer cells cannot be captured as an image depending on environmental factors surrounding the cancer; and the like. Also, because the method of test differs for each cancer, a subject must undergo different clinical tests for each of the cancer tests. Cytodiagnosis is limited to cancers from which samples are easily collected and also covers cancers from which samples can be obtained by endoscope or paracentesis. Nevertheless, it requires medical practitioners with the skill to practice it at a facility equipped with the appropriate medical equipment, and causes the subject pain to no small extent. For tumor markers, blood, a sample that is easy to collect, is used. However, there are problems to be solved, such as that the early stage of cancer cannot be detected; that even an advanced cancer may not reach a high level; and that a high level may be reached due to diseases other than cancer.

Recently, relations between micro RNA (also described as microRNA, miRNA, and the like), which is a single strand nucleic acid with approximately 17-25 bases, and cancers or various diseases, attract attention. It is known that miRNAs have functions to regulate gene expressions, and it has been reported that their types and expression levels in various diseases change from the early stages. In other words, the amount of specific miRNA in patients with cancer increases or decreases as compared to that in healthy individuals; therefore, examination of the amount of concerned miRNA in a sample obtained from a subject becomes a means to know whether the subject has cancer or not. The miRNA exists not only in samples obtained from the body such as tissues and the like but also in body fluids such as urine excreted outside the body, serum, plasma and saliva that can be collected easily. Therefore, use of these three kinds of body fluid samples obtained easily and adoption of miRNA which amount changes when cancer develops as a testing marker, make cancer tests drastically easier to conduct. Furthermore, with a marker that changes regardless of the types of cancers, it is possible to determine whether a subject has cancer or not in a single test that covers all kinds of cancers. If cancer can be found in the early stage easily and inexpensively in physical check-ups and comprehensive medical examinations, it has a significant effect not only by benefiting the subjects but also by reducing medical expenditure.

### BRIEF DESCRIPTION OF DRAWINGS

FIG. 1 is a graph indicating the results from measuring the amount of serum miRNA (SEQ ID NO: 1) obtained from healthy individuals and patients with various cancers using PCR.

FIG. 2 is a graph indicating the results from measuring the amount of serum miRNA (SEQ ID NO: 1) obtained from healthy individuals and patients with various cancers using LAMP.

### DETAILED DESCRIPTION

The embodiments provide a method for providing an index for cancer diagnosis with miRNA as a marker and a kit therefor.

According to the embodiments, miRNA having a nucleotide sequence represented by SEQ ID NO: 1 is used as a marker for cancer diagnosis.

According to the embodiments, a method for distinguishing patients with cancer from healthy individuals by using miRNA in their biological samples as a marker and an assay kit therefor are provided. As the biological sample, a body fluid that is easily obtained can be used, for example, a body fluid such as urine, serum, plasma, saliva, and the like. The miRNA used as a marker in the embodiments has a nucleotide sequence represented by ACUCGGCGUGGCGUCGGUCGUG (SEQ ID NO: 1), and the amount of the RNA in the biological samples obtained from cancer patients is more than that of the RNA in biological samples obtained from healthy individuals.

According to one embodiment, a (in vitro) method for providing an index for diagnosing cancer in a subject and a (in vitro) method for diagnosing cancer in the subject are provided, wherein these methods include (a) a step of measuring the amount of RNA having a nucleotide sequence represented by SEQ ID NO: 1 in a biological sample obtained from the subject, and (b) a step of comparing the measured value obtained in the step (a) to a measured value of the amount of the RNA having the nucleotide sequence represented by SEQ ID NO: 1 measured in the same kind of biological sample obtained from healthy individuals. In these methods, when the measured value in the subject to be measured in the step (a) is higher than the measured value in the healthy individuals, it is indicated that the subject has cancer. On the other hand, when the measured value in the subject to be measured in the step (a) is lower than or equal to the measured value in the healthy individuals, it is indicated that the subject does not have cancer.

According to another embodiment, a kit for diagnosing cancer in a subject is provided. This kit comprises a reagent for measuring the amount of RNA having a nucleotide sequence represented by SEQ ID NO: 1 in the biological sample obtained from the subject.

In the embodiments, a quantitative value of RNA having a nucleotide sequence represented by SEQ ID NO: 1 in the biological sample derived from a subject is compared to the quantitative value in the same kind of biological sample derived from healthy individuals. As a result, when the quantitative value in the subject is higher than that in healthy individuals, it is indicated that the subject has cancer. On the other hand, when the quantitative value in the subject is lower than or equal to that in healthy individuals, it is indicated that the subject does not have cancer.

In the embodiments, a healthy individual means an individual who does not have cancer, preferably an individual who does not have malignant neoplasm including cancer or a malignant tumor. Also, in the embodiments, the quantitative value of RNA having a nucleotide sequence represented by SEQ ID NO: 1 in healthy individuals (measured value of the amount of the RNA) is preferably an upper limit of a proper range that is determined based on the quantitative values in multiple healthy individuals. It is possible to predetermine the proper range based on the data in the past. Furthermore, a threshold can be determined from the viewpoint of the sensitivity and specificity for distinguishing cancer patients from healthy individuals. Based on this threshold, it is possible to judge that a quantitative value in a subject is higher than that in healthy individuals when the quantitative value of the subject is higher than this threshold, and to judge that the quantitative value of the subject is lower than or equal to that in healthy individuals when the quantitative value of the subject is lower than the threshold.

In the embodiments, a subject for diagnosis is preferably a human.

In the embodiments, the type of cancer to be diagnosed is not particularly limited, and any type of cancer wherein miRNA having a nucleotide sequence represented by SEQ ID NO: 1 is highly expressed in a patient who has that type of cancer. Such cancers include, for example, breast cancer, colorectal cancer, gastric cancer, lung cancer, mesothelioma, ovarian cancer, pancreatic cancer, cholangiocarcinoma, esophageal cancer, liver cancer, pharyngeal cancer, maxillary cancer, oral cancer, tongue cancer, lip cancer, laryngeal cancer, thyroid cancer, brain tumor, nerve tumor, glioma, neuroblastoma, renal cell carcinoma, bladder cancer, prostate cancer, testicular cancer, sarcoma, cervical cancer, uterine body cancer, uterine sarcoma, skin cancer, malignant melanoma, bone tumor, leukemia, malignant lymphoma, multiple myeloma, and the like, and preferably at least one kind of cancer selected from the group consisting of breast cancer, colorectal cancer, gastric cancer, lung cancer, ovarian cancer, pancreatic cancer, cholangiocarcinoma, esophageal cancer, liver cancer, brain tumor, bladder cancer, prostate cancer, sarcoma, uterine body cancer, and uterine sarcoma is targeted for the diagnosis.

The biological sample used for measurement is not particularly limited and may be any biological sample in which miRNA expressed in a subject can be detected. Such biological samples include, for example, blood, serum, plasma, leukocytes, urine, digestive juice, saliva, gastric juice, sweat, tear, mucus, sperm, vaginal fluid, amnion fluid, milk, lymphatic fluid, tissue, intraoral mucosa, sputum, and the like. Preferably, a body fluid sample such as serum, plasma, urine, saliva, and the like is used, and more preferably serum or plasma is used. The biological samples may be treated by procedures such as centrifugation, deposition, extraction, and/or separation to be in a suitable form for the amplification of nucleic acids. Also, the biological sample obtained may be used as it is if the biological sample obtained is in a state that is suitable for the amplification of nucleic acids.

In a preferred embodiment, extraction of nucleic acids from the biological sample obtained from a subject is carried out, and the nucleic acids are used as an analysis sample to perform a quantitative measurement (a measurement of the quantity) of miRNA of SEQ ID NO: 1. The extraction of nucleic acids can be easily carried out using commercially available nucleic acid extraction kits. For example, such kits include NucleoSpin (registered trademark) miRNA Plasma (made by Takara Bio Inc.), Quick-cfRNA Serum & Plasma Kit (made by Zymo Research), miRNeasy Serum/Plasma Kit (made by QIAGEN), mirVana PARIS isolation Kit (made by Thermo Fischer Scientific), PureLink™ Total RNA Blood Kit (made by Thermo Fischer Scientific), Plasma/Serum RNA Purification Kit (made by Norgen Biotek Corp.), microRNA Extractor (registered trademark) SP Kit (made by Wako pure chemical Industries, Ltd.), High Pure miRNA Isolation Kit (made by Sigma Aldrich), and the like. Also, instead of using commercially available kits, a simple method of obtaining supernatants from biological samples can also be used, wherein the samples are diluted in a buffer solution and heated at 80-100° C. followed by centrifugation.

The quantitative determination (a measurement of the amount) of miRNA of SEQ ID NO: 1 in the embodiments can be easily carried out by methods known to those skilled in the art. For example, the quantitative determination of miRNA of SEQ ID NO: 1 can be carried out using a nucleic acid amplification technique, and the amount of miRNA can be known by using the speed of amplification as an index. A nucleic acid amplification technique can include PCR, LAMP and the like, and preferably, LAMP can be used. When PCR is used, the amplification speed can be regarded as equivalent to the number of cycles until the signal representing the amount of the amplification product reaches a threshold, and thus measured in terms of the number of cycles. When LAMP is used, the amplification speed can be regarded as equivalent to a time until the signal representing the amount of the amplification product reaches a threshold, and thus measured in terms of the time. It is shown that the expression level of the miRNA will be high when the amplification speed is fast, and the expression level of the miRNA will be low when the amplification speed is slow.

Furthermore, to know the copy number of miRNA of SEQ ID NO: 1 in a biological sample, for example, multiple samples containing the miRNA with a known copy number should be prepared and treated in the similar way to the biological sample from the subject. Subsequently, a calibration curve can be created by performing the quantitative determination of the miRNA. The copy number of the miRNA contained in the biological sample can be known by comparing the measured value in the biological sample from the subject to this calibration curve.

The quantitative determination of miRNA by the PCR can be easily carried out using commercially available kits. Thus, when a qPCR is used, for example, TaqMan Advanced miRNA Assays (made by Thermo Fischer Scientific, ID: 483036_mir), miRCURY LNA miRNA PCR Assays (made by QIAGEN, catalog No. YP02103132), and the like can be used. Also, the qPCR can be performed using SYBR (registered trademark) Green qPCR microRNA detection system (made by OriGene Technologies Inc.) and a specific primer.

When the miRNA is measured by LAMP, the miRNA can be subjected to elongation and LAMP amplification according to a method described in a Japanese Unexamined Patent Application Publication No. 2019-017383. In other words, a first elongation primer which has a sequence that hybridizes with targeted miRNA at the 3′ end and a primer sequence for LAMP amplification or its complementary strand on the 5′ end, and a second elongation primer which has a sequence that hybridizes with a complementary strand of the targeted miRNA at the 3′ end and a primer sequence for the LAMP amplification or its complementary strand at the 5′ end are prepared, and an elongation product is constructed by using these primers for elongation. And then a method of LAMP amplification can be carried out by using LAMP primer sequences contained in the first and second elongation primers.

In that case, a sequence of SEQ ID NO: 2 (CCAGTCCGCCACTGTACTCACGACC) as the first elongation primer, a sequence of SEQ ID NO: 3 (AGGGTTGGAGGTTCATGTCAAGGCCCAGTCTCACGTATTCCACTGACCAC CAGTCGCACTGAGGGGACTCGGCGTGGCGT) as the second elongation primer can be used. For LAMP amplification, amplification can be suitably carried out by using primers represented by SEQ ID NO: 4 (TGGAATACGTGAGACTGGGCCTTTTTTTTTAGGGTTGGAGGTTCATGTCA) and SEQ ID NO: 5 (CTGACCACCAGTCGCACTGAGCCAGTCCGCCACTGTACT) as FIP and BIP primers, respectively and SEQ ID NO: 6 (TGGCGTCGGTC) as a loop primer. Furthermore, a primer having a sequence represented by SEQ ID NO: 7 (CGTCGGTCGTGAG) can also be used as a loop primer.

A kit according to the embodiments can contain the primer and reagent mentioned above as the reagents for measuring the amount of the miRNA. Notably, when the miRNA is assayed using the LAMP, the kit in the embodiment preferably includes a primer set composed of primers each having a nucleotide sequence represented by SEQ ID NOs: 2-6, respectively, or a primer set composed of primers each having a nucleotide sequence represented by SEQ ID NOs: 2-5 and 7, respectively. Furthermore, the kit in the embodiment can appropriately include reagents such as dNTP, a reverse transcriptase, a heat-resistant polymerase, and the like.

Furthermore, the quantitative determination of miRNA using the sets of primers mentioned above is a novel method; therefore, the assay constitutes one embodiment. Therefore, according to one embodiment, a method for measuring the amount of RNA having a nucleotide sequence represented by SEQ ID NO: 1 in a biological sample is provided. And this method comprises nucleic acid amplification by the LAMP using a primer set composed of primers each having a nucleotide sequence represented by SEQ ID NOs: 2-6, respectively, or a primer set composed of primers each having a nucleotide sequence represented by SEQ ID NOs: 2-5 and 7, respectively. Also, according to another embodiment, a kit for measuring the amount of RNA having a nucleotide sequence represented by SEQ ID NO: 1 in a biological sample is provided. And this kit comprises a primer set composed of primers each having a nucleotide sequence represented by SEQ ID NOs: 2-6, respectively, or a primer set composed of primers each having a nucleotide sequence represented by SEQ ID NOs: 2-5 and 7, respectively. This kit can also include other reagents used for the LAMP.

According to the embodiments, it is possible to determine whether a subject has cancer or not. There are a variety of known cancer-related diagnoses using miRNA as a marker, for example, such as those for predicting the stage of specific cancer, and those for predicting cancer recurrence and prognosis. However, the diagnosis according to the embodiment is different from those. Also, according to the embodiments, it is possible to carry out a diagnosis for any cancer, particularly multiple types of cancers, and not a sole specific cancer. Notably, it may be said that this is suitable for the primary screening that covers a wide range of cancers, for example, in annual physical check-ups and the like.

The above-described embodiments are examples, and the scope of the invention should not be limited to those.

### EXAMPLES

The embodiments will be explained specifically according to the following examples, but the scope of the inventions is not limited to these examples.

In the following examples, “sensitivity,” “specificity,” and “AUC”, indicating the precision of the examination, have the following meanings. In cases in which the test value is determined to be positive when exceeding a particular threshold value, “sensitivity” is a numerical value from 0 to 1 expressing the ratio of the test value exceeding the threshold value among the test values of the disease group. Therefore, results closer to the sensitivity of 1 indicate less false-negative. In cases in which the test value is determined to be positive when exceeding a particular threshold value, “specificity” is a numerical value from 0 to 1 expressing the ratio of the test value lower than or equal to the threshold value among the test values of the healthy group. Therefore, results closer to the specificity of 1 indicate less false-positive. Finally, “AUC” represents the area under the curve of a ROC curve, which is made by setting sensitivity as the vertical axis (0-1) and (1-specificity) as the horizontal axis (0-1) while the threshold is continually changed. The closer the AUC is to 1 (in other words, closer the curve is to the upper left corner) shows that the diagnostic capability of the diagnostic method is high. On the contrary, the closer the AUC is to 0.5 (in other words, closer the curve is to the diagonal line) shows that the diagnostic capability of the diagnostic method is low.

### Example 1: Quantitative Determination of miRNA by qRT-PCR

In the present example, the quantitative determination of miRNA of SEQ ID NO: 1 was performed in serum from healthy individuals and cancer patients using a qRT-PCR.

There were 16 sample specimens from healthy individuals and 94 sample specimens from cancer patients; the sample specimens from cancer patients break down as 22 cases of breast cancer, 6 cases of colorectal cancer, 6 cases of gastric cancer, 3 cases of lung cancer, 6 cases of ovarian cancer, 6 cases of pancreatic cancer, 5 cases of cholangiocarcinoma, 5 cases of esophageal cancer, 5 cases of liver cancer, 5 cases of brain tumor, 5 cases of bladder cancer, 5 cases of prostate cancer, 5 cases of sarcoma, 5 cases of uterine body cancer and 5 cases of uterine sarcoma. All the serum was extracted using NucleoSpin (registered trademark) miRNA Plasma (Takara Bio Inc.). According to the instruction manual of TaqMan (registered trademark) Advanced miRNA Assay (Thermo Fischer Scientific), cDNA was synthesized using TaqMan (registered trademark) Advanced miRNA cDNA Synthesis Kit (Thermo Fischer Scientific). Subsequently, TaqMan PCR was performed using TaqMan (registered trademark) Fast Advanced Master Mix (Thermo Fischer Scientific) and TaqMan (registered trademark) Advanced miRNA Assay ID: 483036_mir, and Ct levels were measured. In addition to the samples, 10³, 10⁴, 10⁵, 10⁶ copies/μL of the synthetic RNA of SEQ ID NO: 1 were similarly reacted, and a calibration curve was made. The result for 10² copies/μL of the RNA was below the detection lower limit.

The results are depicted in Table 1 below.

TABLE 1
Results of PCR

| Cancer Type | AUC |
|---|---|
| Breast cancer | 0.92 |
| Pancreatic cancer | 0.97 |
| Ovarian cancer | 0.99 |
| Uterine cancer | 0.97 |
| Uterine sarcoma | 0.97 |
| Prostate cancer | 0.99 |
| Esophageal cancer | 0.86 |
| Gastric cancer | 0.98 |
| Colorectal cancer | 1.00 |
| Liver cancer | 0.87 |
| Cholangiocarcinoma | 0.93 |
| Bladder cancer | 1.00 |
| Lung cancer | 1.00 |
| Brain tumor | 1.00 |
| Sarcoma | 0.95 |
| All cancers | 0.95 |

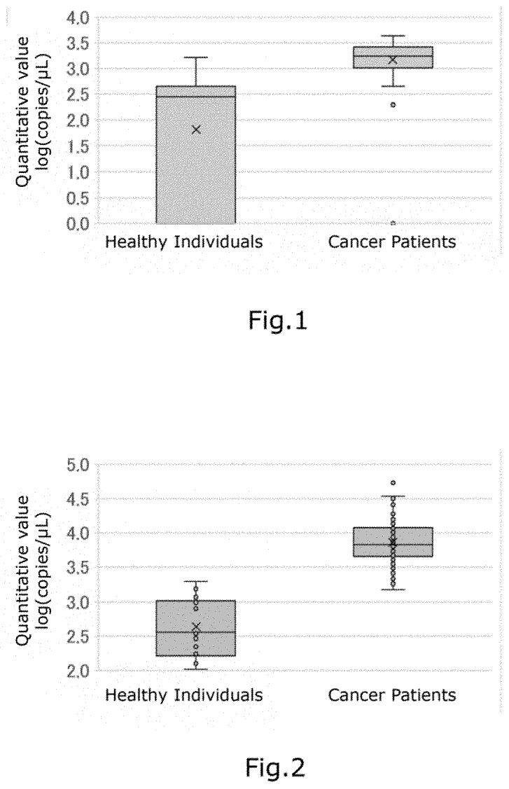

As a result of the quantitative determination, four sample specimens from the healthy individuals were below the detection lower limit. Also, most of the sample specimens from the healthy individuals which was detectable exhibited quantitative values lower than 10³ copies/μL, which was the lower limit of the calibration curve, and thus the calibration curve was extrapolated to obtain estimated values which were of the order of 10² copies/μL. On the other hand, many of the quantitative values from the cancer patients exceeded 10³ copies/μL regardless of the types of cancer. The quantitative determination results of 110 sample specimens were shown in the figure of the box-and-whisker plot (FIG. 1). The vertical axis indicates the copy number per 1 μL of the extract by the logarithm to base 10. It is shown that the values obtained from cancer patients were distributed at a higher level than those obtained from healthy individuals. When the threshold was set at 10³ copies/μL, the sensitivity, specificity, and the AUC (Area Under Curve), which differentiated healthy individuals from cancer patients, was 0.83, 0.94, and 0.95, respectively. Thus, it was shown that miRNA of SEQ ID NO: 1 had high-performance as a marker for determination of cancer.

### Example 2: Quantitative Determination of miRNA by LAMP

In the present example, the quantitative determination of miRNA of SEQ ID NO: 1 was performed in serum from healthy individuals and cancer patients using LAMP.

A breakdown of sample specimens and an extraction method are the same as those used in Example 1. 2 μL of the extracted sample was reverse-transcripted under conditions of 20 μL in a reaction volume, at 16° C. for 10 minutes, at 42° C. for 5 minutes and at 85° C. for 5 minutes. The composition of the reverse transcription reaction solution included 67 unit MultiScribe (registered trademark) Reverse Transcriptase (*), 1× RT Buffer (*), 0.1 mM dNTPs (*), 4 U RNaseOUT (Thermo Fischer Scientific), and 10 nM RT primer (CCAGTCCGCCACTGTACTCACGACC: SEQ ID NO: 2). As for *, the items contained in High-Capacity cDNA Reverse Transcription Kit (Thermo Fischer Scientific) were used. To the reaction solution after the reverse-transcription, 5 μL of an elongation solution was added. After the reaction solution was treated at 95° C. for 2 minutes, the elongation reaction was carried out by performing 20 cycles (one cycle consists of reactions at 95° C. for 20 seconds, at 59° C. for 30 seconds, and at 72° C. for 10 seconds). As for the elongation solution to be added, 0.5 U Deep Vent (exo-) DNA polymerase (New England Bio) was contained in 25 μL of the elongation solution, and the reaction solution was adjusted so that the final concentrations were 0.2× ThermoPol Buffer (Deep Vent (exo-) DNA polymerase, attached), 0.2 mM MgSO₄, 0.12 mM dNTPs, and 10 nM EL primer (AGGGTTGGAGGTTCATGTCAAGGCCCAGTCTCACGTATTCCACTGACCAC CAGTCGCACTGAGGGGACTCGGCGTGGCGT: SEQ ID NO: 3). Next, LAMP amplification was carried out for 1 μL of the elongation product under the conditions of the reaction volume of 25 μL, at a temperature of 65° C. for 60 minutes, and the rise time of the fluorescence intensity was measured. The LAMP amplification solution included 8 U Tin (exo-) LF DNA polymerase (Optigene), 0.5 μL EvaGreen (registered trademark) (Biotium), and the reaction solution was adjusted so that the final concentrations were 20 mM Tris-HCl (pH 8.0), 50 mM KCl, 8 mM MgSO₄, 10 mM (NH₄)SO₄, 0.1% Tween-20, 0.8 M betaine, 1.4 mM each dNTPs, 1.6 μM FIP primer (TGGAATACGTGAGACTGGGCCTTTTTTTTTAGGGTTGGAGGTTCATGTCA: SEQ ID NO: 4), 1.6 μM BIP primer (CTGACCACCAGTCGCACTGAGCCAGTCCGCCACTGTACT: SEQ ID NO: 5), and 0.8 μM LB primer (TGGCGTCGGTC: SEQ ID NO: 6), respectively. The fluorescence intensity was measured over time with a real-time PCR device, and time until the intensity exceeded the threshold was measured. Similarly, samples were reacted with 10², 10³, 10⁴, 10⁵ copies/μL of the synthetic RNA of SEQ ID NO: 1, and a calibration curve was made.

The results are depicted in Table 2 below.

TABLE 2
Results of LAMP

| Cancer Type | AUC |
|---|---|
| Breast cancer | 0.99 |
| Pancreatic cancer | 1.00 |
| Ovarian cancer | 1.00 |
| Uterine cancer | 1.00 |
| Uterine sarcoma | 1.00 |
| Prostate cancer | 1.00 |
| Esophageal cancer | 1.00 |
| Gastric cancer | 1.00 |
| Colorectal cancer | 1.00 |
| Liver cancer | 1.00 |
| Cholangiocarcinoma | 1.00 |
| Bladder cancer | 0.98 |
| Lung cancer | 1.00 |
| Brain tumor | 1.00 |
| Sarcoma | 1.00 |
| All cancers | 0.99 |

As a result of the quantitative determination, the quantitative values were distributed at the order of 10²⁻³ copies/μL for the healthy individuals, and at the order of 10³⁻⁴ copies/μL for the cancer patients regardless of the types of cancers. The quantitative determination results of 110 sample specimens were shown in the figure of the box-and-whisker plot (FIG. 2). It is shown that values obtained from cancer patients were distributed at a higher level than those obtained from healthy individuals. When the threshold was set at 10³ copies/μL, the sensitivity, specificity, and the AUC (Area Under Curve), which differentiated healthy individuals from cancer patients, were 0.95, 1.00, and 0.99, respectively. Thus, it was shown that miRNA of SEQ ID NO: 1 had high-performance as a marker for determination of cancer.

### Example 3: Examination when a Loop Primer of SEO ID NO: 7 was Used Instead of a Loop Primer of SEQ ID NO: 6

When the elongation product for a calibration curve by a synthetic RNA synthesized in Example 2 was amplified by LB of SEQ ID NO: 7 (CGTCGGTCGTGAG) instead of LB of SEQ ID NO: 6, a similar calibration curve was obtained. Thus, LB of SEQ ID NO: 7 was shown to be effective in the present LAMP amplification system.

While certain embodiments have been described, these embodiments have been presented by way of example only, and are not intended to limit the scope of the inventions. Indeed, the novel methods and products described herein may be embodied in a variety of other forms; furthermore, various omissions, substitutions and changes in the form of the methods and products described herein may be made without departing from the spirit of the inventions. The accompanying claims and their equivalents are intended to cover such forms or modifications as would fall within the scope and spirit of the inventions.

1. A method for diagnosing cancer in a subject, comprising the steps of:
(a) measuring the amount of RNA having a nucleotide sequence represented by SEQ ID NO: 1 in a biological sample obtained from the subject, and
(b) comparing the measured value obtained in the step (a) to a measured value of the amount of the RNA having the nucleotide sequence represented by SEQ ID NO: 1 measured in the same kind of biological sample obtained from healthy individuals,
wherein, it is indicated that the subject has cancer when the measured value in the subject to be measured in the step (a) is higher than the measured value in the healthy individuals, and that the subject does not have cancer when the measured value in the subject to be measured in the step (a) is lower than or equal to the measured value in the healthy individuals, and
wherein the cancer is at least one type of cancer selected from the group consisting of breast cancer, colorectal cancer, gastric cancer, lung cancer, ovarian cancer, pancreatic cancer, cholangiocarcinoma, esophageal cancer, liver cancer, brain tumor, bladder cancer, prostate cancer, sarcoma, uterine body cancer, and uterine sarcoma.

2. The method according to claim 1, wherein the biological sample is serum or plasma.

3. The method according to claim 1, wherein the measured value in the healthy individual is an upper limit of a proper range that is determined based on the quantitative values in multiple healthy individuals.

4. The method according to claim 1, wherein the measurement of the amount of the RNA in a biological sample is performed using a nucleic acid amplification technique.

5. The method according to claim 4, wherein the nucleic acid amplification technique is PCR.

6. The method according to claim 4, wherein the nucleic acid amplification technique is LAMP.

7. The method according to claim 6, wherein the LAMP is performed using a primer set composed of primers each having a nucleotide sequence represented by SEQ ID NOs: 2-6, respectively.

8. The method according to claim 6, wherein the LAMP is performed using a primer set composed of primers each having a nucleotide sequence represented by SEQ ID NOs: 2-5 and 7, respectively.

9. The method according to claim 1, wherein a copy number of the RNA in a biological sample is determined by a calibration curve that is made based on multiple samples containing a known copy number of the RNA.

10. A kit for diagnosing cancer in a subject, comprising a reagent for measuring the amount of RNA having a nucleotide sequence represented by SEQ ID NO: 1 in a biological sample obtained from the subject,
wherein the cancer is at least one type of cancer selected from the group consisting of breast cancer, colorectal cancer, gastric cancer, lung cancer, ovarian cancer, pancreatic cancer, cholangiocarcinoma, esophageal cancer, liver cancer, brain tumor, bladder cancer, prostate cancer, sarcoma, uterine body cancer, and uterine sarcoma.

11. The kit according to claim 10, wherein the biological sample is serum or plasma.

12. The kit according to claim 10, wherein the reagent is a reagent for a nucleic acid amplification technique.

13. The kit according to claim 12, wherein the nucleic acid amplification technique is PCR.

14. The kit according to claim 12, wherein the nucleic acid amplification technique is LAMP.

15. The kit according to claim 14, wherein the kit comprises a primer set composed of primers each having a nucleotide sequence represented by SEQ ID NOs: 2-6, respectively.

16. The kit according to claim 14, wherein the kit comprises a primer set composed of primers each having a nucleotide sequence represented by SEQ ID NOs: 2-5 and 7, respectively.

17. A method for measuring the amount of RNA having a nucleotide sequence represented by SEQ ID NO: 1 in a biological sample, comprising performing nucleic acid amplification by LAMP using a primer set composed of primers each having a nucleotide sequence represented by SEQ ID NOs: 2-6, respectively, or a primer set composed of primers each having a nucleotide sequence represented by SEQ ID NOs: 2-5 and 7, respectively.

18. A kit for measuring the amount of RNA having a nucleotide sequence represented by SEQ ID NO: 1 in a biological sample, comprising a primer set composed of primers each having a nucleotide sequence represented by SEQ ID NOs: 2-6, respectively, or a primer set composed of primers each having a nucleotide sequence represented by SEQ ID NOs: 2-5 and 7, respectively.
